(19) United States
(12) Patent Application Publication
(10) Pub. No.: US 2018/0161045 A1
(43) Pub. Date: Jun. 14, 2018

(54) CUTTER

(71) Applicant: L.A.R.S.-LABORATOIRE D'APPLICATION ET DE RECHERCHE SCIENTIFIQUE, Arc Sur Tille (FR)

(72) Inventors: Bernard Brulez, Bourbonne Les Bains (FR); Gérard Berbey, Barges (FR); Giuliano Cerulli, Perugia (IT)

(73) Assignee: L.A.R.S.-LABORATOIRE D'APPLICATION ET DE RECHERCHE SCIENTIFIQUE, Arc Sur Tille (FR)

(21) Appl. No.: 15/571,074

(22) PCT Filed: May 12, 2016
(86) PCT No.: PCT/FR2016/051128

§ 371 Date: Nov. 1, 2017

(30) Foreign Application Priority Data
May 12, 2015 (FR) 15 54239

(51) Int. Cl. A61B 17/16

(52) U.S. Cl. CPC A61B 17/1617; A61B 2017/00986; A61B 17/1624

(57) ABSTRACT

A surgical cutter intended in particular for the prosthetic replacement of a knee ligament and comprising a blade-holder tube provided with a handle at one of its ends, a blade of generally rectangular shape mounted pivotably, at the other end of said blade-holder tube, about a rotation axis perpendicular to the longitudinal axis of the latter between a “retracted” position, in which it does not protrude from the body of said blade-holder tube, and a “deployed” position, in which it extends on each side of said blade-holder tube, and a locking mechanism for locking said blade in the “retracted” position or “deployed” position.

### FIELD OF THE INVENTION

The present invention relates to a multipurpose surgical instrument particularly for the plasty (or prosthetic replacement) of a knee ligament.

### PRIOR ART

In the case of a ligament rupture, following an accident for example, it is well-known that it may be necessary to replace the damaged ligament(s) which, in the case of some, cannot regenerate themselves.

The difficulty in replacing this type of ligament is no longer associated with the ligaments themselves since very suitable artificial ligament embodiments are now known, but with restricted accessibility problems due to the particularly overcrowded anatomy of the joints, as well as with problems associated with artificial ligament holders, i.e. the anatomic parts wherein and on said ligament is sought to be mounted.

Indeed, in order to mount and insert the ligament, it is necessary to produce tunnels (drill holes) in some anatomical parts such as the femur and tibia. However, in view of the mechanical strength of these anatomical parts, it is important to minimise the number and dimensions of these tunnels, particularly the diameter thereof, so as not to weaken said anatomical parts.

### DESCRIPTION OF THE INVENTION

Therefore, the aim of the present invention is that of remedying these drawbacks by providing a multipurpose surgical instrument, used in particular for the plasty (or prosthetic replacement) of a knee ligament, of compact design, easy to use and suitable for only increasing the diameter of a tunnel produced in an anatomical part locally, particularly and essentially at the inaccessible end of said tunnel opening into a joint cavity.

In this regard, the present invention relates to a surgical cutter intended in particular for the plasty (or prosthetic replacement) of a knee ligament and comprising:
a blade-holder tube provided with a handle at one of the ends thereof,
a blade of generally rectangular shape mounted pivotably, at the other end of said blade-holder tube, about a rotation axis perpendicular to the longitudinal axis thereof between a “retracted” position wherein it does not protrude from the body of said blade-holder, and a “deployed” position, and
locking means suitable for locking said blade in the “retracted” position or “deployed position, said cutter being characterised in that the blade rotates freely when it is not locked by the locking means and in that it extends on either side of said blade-holder tube when it is in the “deployed” position.

Advantageously, the blade-holder tube is a hollow tube and comprises:
a slot traversing therethrough and suitable for receiving the blade when it is oriented such that the longitudinal axis thereof is aligned with the longitudinal axis of said blade-holder tube, and
an orifice traversing therethrough and being arranged such that the longitudinal axis thereof is perpendicular to the longitudinal plane of symmetry of the slot and passes through the centre of said slot, said orifice being suitable for engaging with a rivet to enable the blade to pivot about the longitudinal axis thereof.

The blade is preferably a sheet of generally rectangular shape wherein the thickness is strictly less than the thickness of the slot of the blade-holder tube, the width is at most equal to the diameter D of the blade-holder tube and the length is strictly greater than said diameter D, and said blade further comprises a central orifice traversing through the thickness thereof and suitable for engaging with the rivet to enable the pivoting thereof about the longitudinal axis of said orifice.

According to one preferred embodiment, the locking means comprise a shaft wherein one of the ends is suitable for engaging with the blade and a locking member fastened to the other end of said shaft, suitable for engaging with the end of the blade-holder tube, opposite that receiving the blade, in order to slide said shaft inside the blade-holder tube and lock same in a defined position.

The blade advantageously comprises cut-out sections situated at the midpoint of the lateral and longitudinal edges thereof.

The end of the shaft is then suitable for engaging with one of the cut-out sections of the blade.

Preferably, the locking member is rigidly connected to the end of the blade-holder tube by a helical link and in that it comprises a cap provided with an internal or external thread suitable for engaging with an internal or external thread produced on the associated end of the blade-holder tube.

### BRIEF DESCRIPTION OF THE FIGURES

Further advantages and features will emerge more clearly from the following description of an alternative embodiment of a surgical cutter with reference to the appended drawings wherein:

FIG. 1 is an overall view of a surgical cutter according to the invention with the blade thereof in the “deployed” position;

FIG. 2 is an enlarged detailed view of FIG. 1;

FIG. 3 is a detailed view along the direction F in FIG. 2;

FIGS. 4 to 6 are enlarged and partial cross-sectional views of the surgical cutter in FIG. 1 at various stages of the use thereof.

### PREFERRED EMBODIMENT OF THE TECHNICAL INVENTION

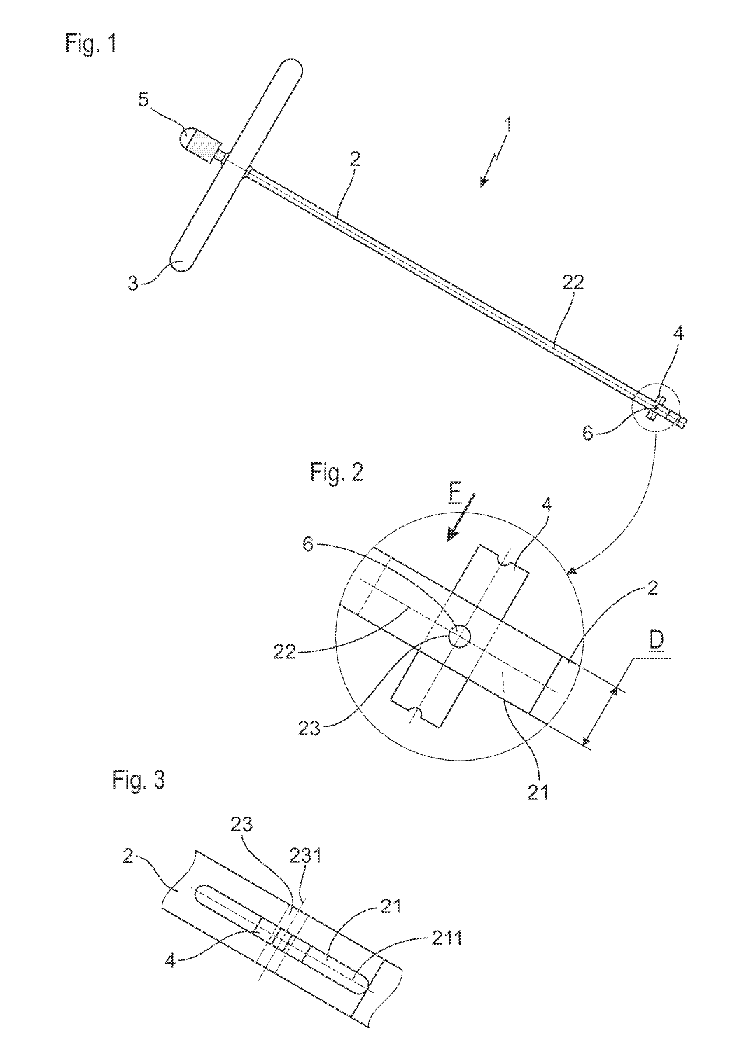

In FIGS. 1 to 6, a surgical cutter 1, according to the invention, has been represented, comprising a hollow blade-holder tube 2 provided at one of the ends thereof with a handle 3, a flat blade 4 of generally rectangular shape mounted pivotably at the other end of said blade-holder tube 2 about an axis of rotation perpendicular to the longitudinal axis thereof and locking means 5 suitable for locking said blade 4 in position.

The term “generally rectangular” denotes herein a shape which may be rectangular in the strict sense with a right angle at each vertex or indeed a rectangular shape with a rounded corner at each vertex optionally ranging up to an oblong shape.

The blade-holder tube 2, which extends beyond the handle 3, is a tube with a circular cross-section and comprises:
a slot 21 traversing therethrough and suitable for receiving the blade 4 when it is oriented such that the longitudinal axis thereof is aligned with the longitudinal axis 22 of said blade-holder tube 2, and
an orifice 23 traversing therethrough and being arranged such that the longitudinal axis 231 thereof is perpendicular to the longitudinal plane 211 of symmetry of the slot 21 and passes through the centre of said slot 21.

Said orifice 23 being suitable for engaging with a rivet 6 to enable the blade 4 to pivot about the longitudinal axis 231 thereof.

The blade 4 is a sheet of generally rectangular shape wherein the thickness is strictly less than the thickness of the slot 21 of the blade-holder tube 2, the width is at most equal to the diameter D of the blade-holder tube 2 and the length is strictly greater than said diameter D.

The blade-holder tube may have a non-circular cross-section without leaving the scope of the present invention, the diameter D will then correspond to the size of said blade-holder tube 2 situated in the longitudinal plane of symmetry of said slot.

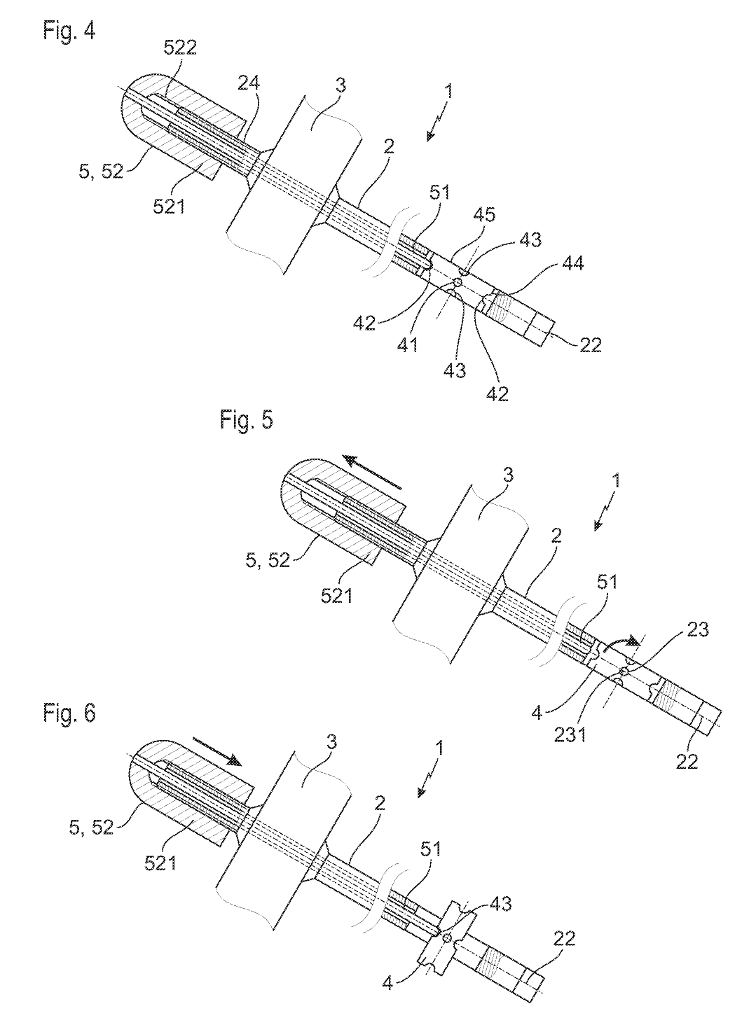

Said blade 4 further comprises a central orifice 41 traversing through the thickness thereof and suitable for engaging with the rivet 6 to enable the pivoting thereof about the longitudinal axis 231 of said orifice 23 between a “retracted” position wherein the blade 4 is oriented such that the longitudinal axis thereof is aligned with the longitudinal axis 22 of said blade-holder tube 2 (see FIG. 4) and a “deployed” position wherein the blade 4 is oriented such that the longitudinal axis thereof is perpendicular to said longitudinal axis 22 (see FIG. 6), the blade 4 only protruding from the blade-holder tube 2 when it is in the “deployed” position wherein it extends on either side of said blade-holder tube 2.

Moreover, the blade 4 also comprises cut-out sections 42, 43 situated at the midpoint respectively of the lateral 44 and longitudinal 45 edges thereof, said cut-out sections 42, 43 being preferably semicircular in shape. However, said cut-out sections 42, 43 may be of any other shape such as, for example, square, rectangular or triangular, without leaving the scope of the present invention.

The cutter 1 according to the invention also comprises locking means 5 suitable for locking said blade 4 in the “retracted” position or “deployed position. Said locking means 5 comprise a shaft 51 wherein one of the ends is suitable for engaging with the cut-out sections 42, 43 of the blade 4 and a locking member 52 fastened to the other end of said shaft 51, suitable for engaging with the end of the blade-holder tube 2, opposite that receiving the blade 4, in order to slide said shaft 51 inside the blade-holder tube 2 and lock same in a defined position.

Said locking member 52 is therefore rigidly connected to the end of the tube-holder blade 2 by a link enabling the relative displacement of the locking member 52 with respect to the blade-holder tube 2 at least along the longitudinal axis thereof. For reasons of ease of cleaning, said link is preferably a helical link and the locking member 52 is in this case a cap 521 provided with an internal thread 522 suitable for engaging with an external thread 24 produced on the associated end of the blade-holder tube 2.

The cap 521 of the locking member 52 may be provided with an external thread suitable for engaging with an external thread produced on the associated end of the blade-holder tube 2, without leaving the scope of the present invention.

As such, with reference to FIGS. 4 to 6 and with this surgical cutter 1 design, according to the invention, the procedure described hereinafter is applied to produce a counterbore in an existing tunnel (or pre-tunnel) so as to increase the diameter of said tunnel locally.

First of all, after having produced a pre-tunnel of a slightly greater diameter than the diameter of the blade-holder tube 2 in the tibia and the femur, observing a correct alignment with no angulation, the femoral tunnel, followed by the tibial tunnel, will be prepared.

For this, under arthroscopic monitoring, the blade-holder tube 2 of the cutter 1, wherein the blade 4 is locked in the “retracted” position by the locking means 5, is inserted.

Then, when the end of the cutter 1 is in the joint cavity, insertion is continued by approximately some ten millimetres beyond the end of the pre-tunnel opening into said cavity.

At this stage, the blade 4 is released by unfastening the cap 521 of the locking member 52 slightly from the associated end of the blade-holder tube 2 such that the end of the shaft 51 is no longer engaged in the associated cut-out section 42, conventionally by a rotation in the anti-clockwise direction.

Then, using a spindle or optionally an arthroscopic instrument, the blade 4 is pivoted about the longitudinal axis 231 of the orifice 23 of the blade-holder tube 2 until it reaches the “deployed” position.

Next, the blade 4 is locked in this position by refastening the cap 521 of the locking member 52 onto the associated end of the blade-holder tube 2 such that the end of the shaft 51 is engaged into the associated cut-out section 43, conventionally by a rotation in the clockwise direction.

Finally, the cutter 1 is pulled outwards, using the handle 3 by making rotation movements, thereby boring the tunnel of a length and a diameter suitable for the chosen surgical procedure.

Finally, the blade 4 is unlocked once again, pivoted to return it to the “retracted” position thereof, locked once again and removed from the femoral tunnel.

If required, after selecting the cutter, the same procedure is applied to produce the tibial tunnel to dimensions suitable for the chosen surgical procedure.

### INDUSTRIAL APPLICABILITY

It is understood that the cutter 1, according to the invention, is more particularly suitable for the plasty (or prosthetic replacement) of a knee ligament, but it may also be used for other types of surgical procedure.

Finally, it is obvious that the present invention is not restricted to the sole embodiment of this cutter 1; on the contrary, it encompasses all alternative embodiments and applications observing the same principle.

1-8. (canceled)

9. A surgical cutter intended in particular for the plasty, or prosthetic replacement of a knee ligament and comprising:
a blade-holder tube provided with a handle at one of the ends thereof,
a blade of generally rectangular shape mounted pivotably, at the other end of said blade-holder tube, about a rotation axis perpendicular to the longitudinal axis thereof between a retracted position wherein it does not protrude from the body of said blade-holder, and a deployed position, and
locking means suitable for locking said blade in the retracted position or deployed position, wherein the blade rotates freely when it is not locked by the locking means and it extends on either side of said blade-holder tube when it is in the deployed position.

10. The cutter according to claim 9, wherein the blade-holder tube is a hollow tube and comprises: a slot traversing therethrough and suitable for receiving the blade when it is oriented such that the longitudinal axis thereof is aligned with the longitudinal axis of said blade-holder tube, and an orifice traversing therethrough and being arranged such that the longitudinal axis thereof is perpendicular to the longitudinal plane of symmetry of the slot and passes through the centre of said slot, said orifice being suitable for engaging with a rivet to enable the blade to pivot about the longitudinal axis thereof.

11. The cutter according to claim 10, wherein the blade is a sheet of generally rectangular shape wherein the thickness is strictly less than the thickness of the slot of the blade-holder tube, the width is at most equal to the diameter D of the blade-holder tube and the length is strictly greater than said diameter D, and the blade further includes a central orifice traversing through the thickness thereof and suitable for engaging with the rivet to enable the pivoting thereof about the longitudinal axis of said orifice.

12. The cutter according to claim 9, wherein the locking means comprise a shaft wherein one of the ends is suitable for engaging with the blade and a locking member fastened to the other end of said shaft, suitable for engaging with the end of the blade-holder tube, opposite that receiving the blade, in order to slide said shaft inside the blade-holder tube and lock same in a defined position.

13. The cutter according to claim 9,wherein the blade includes cut-out sections situated at the midpoint respectively of the lateral and longitudinal edges thereof.

14. The cutter according to claim 13, wherein said cut-out sections are semicircular in shape.

15. The cutter according to claim 14, wherein the end of the shaft is suitable for being engaged in one of the cut-out sections of the blade.

16. The cutter according to claim 12, wherein the locking member is rigidly connected to the end of the blade-holder tube by a helical link and in that the locking member includes a cap provided with an internal thread or external thread suitable for engaging with an external thread or internal thread produced on the associated end of the blade-holder tube.
